(19) United States
(12) Patent Application Publication
(10) Pub. No.: US 2008/0286350 A1
(43) Pub. Date: Nov. 20, 2008

(54) Formulation of Multivalent Antibody Constructs and Use of Same for Cancer Therapy

(75) Inventors: Marcel Bally, Bowen Island (CA); Gigi Chiu, Richmond (CA)

Correspondence Address: OYEN, WIGGS, GREEN & MUTALA LLP;480 - THE STATION, 601 WEST CORDOVA STREET, VANCOUVER, BC, V6B 1G1, CA

(73) Assignee: BC CANCER AGENCY, Vancouver, BC (CA)

(21) Appl. No.: 11/576,596

(22) PCT Filed: Oct. 6, 2005
(86) PCT No.: PCT/CA05/01535

§ 371 Date: Nov. 30, 2007

Related U.S. Application Data
(60) Provisional application No. 60/615,944, filed on Oct. 6, 2004.

(51) Int. Cl. A61K 9/127; G01N 33/53; G01N 33/574; A61P 31/00; A61K 39/395; A01K 67/027

(52) U.S. Cl. 424/450; 435/7.2; 435/7.23; 800/3; 424/178.1

(57) ABSTRACT

The invention relates to the formation of multivalent antibody constructs for testing and therapeutic purposes. In one embodiment the constructs consist of antibodies or antibody fragments conjugated to liposomes. The constructs are employed in a cell-based in vitro assay for comparing the therapeutic activity of antibodies or antibody fragments in multivalent form to the same antibodies or fragments in bivalent, free form. The assay is useful for identifying antibodies having potential in vivo activity. Selected antibodies may then be tested in an animal model of a disease state, such as cancer or an autoimmune disorder. Co-delivery of antibodies and chemotherapeutics may also be investigated. In accordance with the invention, a significant enhancement in the activity of antibodies such as trastuzumab and rituximab was observed when these antibodies were presented in the multivalent liposomal form. Key cell survival signaling molecules were down-regulated upon treatment with the multivalent liposomal antibody construct. The invention demonstrates the potential of liposome technology to enhance the therapeutic effect of antibodies via a mechanism that modulates cell survival, likely through clustering of target/antibody complex.

### REFERENCE TO RELATED APPLICATIONS

This application claims the benefit of U.S. provisional patent application Ser. No. 60/615,944 filed 6 Oct. 2004 which is hereby incorporated by reference.

### FIELD OF THE INVENTION

The present invention relates to the formulation and therapeutic use of multivalent antibody constructs, such as antibodies or antibody fragments conjugated to liposomes.

### BACKGROUND OF THE INVENTION

Monoclonal antibodies have become the most rapidly expanding class of pharmaceuticals for treating a wide range of human diseases, including cancer. All clinically approved and most experimental antibody drugs directly target tumour cells. Several strategies are being explored to increase the efficacy of such antibodies, including enhancement of effector functions, direct and indirect arming and pre-targeting of prodrugs or radionucleotides ¹. One such strategy is to couple the antibody to a liposome in order to provide a form of the antibody that is more effective and has altered absorption, metabolism, excretion and toxicity.

Liposomes are microscopic particles that are made up of one or more lipid bilayers enclosing an internal compartment. Liposomes can be categorized into multilamellar vesicles, multivesicular liposomes, unilamellar vesicles and giant liposomes. Liposomes have been widely used as carriers for a variety of agents such as drugs, cosmetics, diagnostic reagents, and genetic material. Since liposomes consist of non-toxic lipids, they generally have low toxicity and therefore are useful in a variety of pharmaceutical applications. In particular, liposomes are useful for increasing the circulation lifetime of agents that have a short half-life in the bloodstream. Liposome encapsulated drugs often have biodistributions and toxicities which differ greatly from those of free drug. For specific in vivo delivery, the sizes, charges and surface properties of these carriers can be changed by varying the preparation methods and by tailoring the lipid makeup of the carrier. For instance, liposomes may be made to release a drug more quickly by decreasing the acyl chain length of a lipid making up the carrier.

Monoclonal antibodies have previously been coupled to the surface of liposomes as a method of selectively targeting liposomes to sites of disease. However, such antibodies do not necessarily provide any therapeutic effect and typically the therapeutic drug is encapsulated within the liposomes. The need has therefore arisen for improved methods for identifying antibodies or antibody fragments likely to have a therapeutic effect and for enhancing that effect in vivo.

The therapeutic effectiveness of antibodies can be attributed to multiple functional properties of these molecules, including ligand binding competition, antibody-dependent cellular cytotoxicity (ADCC), complement-dependent cytotoxicity (CDC), interference with receptor dimerization and signaling, and induction of apoptosis ¹⁻³. Various strategies have been explored in the prior art to increase the efficacy of antibody-based therapies, including (1) modifications to the Fc domain to enhance effector functions for improving ADCC and CDC responses, (2) modifications to the binding domain to increase affinity for target antigens, (3) attachment of toxins, drugs, radionuclides or cytokines to the antibody, (4) pre-targeting of pro-drugs and radionuclides to diminish systemic toxicities of these agents, and (5) construction of multivalent antibodies or fragments via chemical conjugation or protein expression ¹.

Of the various strategies, multivalent antibody constructs are of particular interest. These complexes have considerable therapeutic potential and may form the basis for development of cancer targeted nanopharmaceuticals. Many of the tumor antigens to which therapeutic antibodies have been developed function through dimerization or exist as clusters of tens to hundreds of molecules on cell surface ^{4, 5} When these tumor antigens are exposed to multivalent constructs of antibodies or fragments, clustering of the target/antibody complex occurs as a result of increased valency and avidity of the construct. It has been shown that multimerization of antibodies or fragments can decrease off-rate and increase the efficiency of inducing clustering of the target/Ab complex ⁶. Some studies have shown that cross-linking or multimerization of several antibodies or fragments enhanced biological responses as compared to the bivalent format. These responses include activation of cell death signals, inhibition of cell survival signals and/or increased internalization of the tumor antigen ⁷⁻¹⁰. This is exemplified by studies with rituximab, a therapeutic antibody targeting CD20. Under in vivo conditions, the Fc_{γ}R-expressing cells are thought to act as cross-linking agents that induce the clustering of CD20/rituximab and subsequent apoptotic signals, in addition to their role in mediating ADCC ^{2, 7, 8}. Currently, multimerization of Ab or fragments can be achieved through a number of techniques; however, not all of the approaches can be applied in vivo. For instance, the use of anti-IgG Ab or Protein A/G as cross-linking agents is not suitable for in vivo use, although these agents are commonly used in vitro because of ease and simplicity. Alternatively, multivalent Ab or fragments can be readily generated by protein expression technology; however, in vivo application of these constructs often requires a fine tuning of various parameters including tumor penetrability, target binding affinity, size and circulation longevity of the construct ^{6, 11}.

The need has therefore arisen for improved methods for identifying antibodies having therapeutic potential and for formulating and using constructs employing such antibodies for testing and therapeutic purposes.

### SUMMARY OF THE INVENTION

The invention relates to a method for the identification of therapeutically active antibodies. In one embodiment the method includes the steps of formulating a multivalent antibody construct comprising a plurality of antibodies or antibody fragments; measuring the therapeutic activity of the construct in vitro; and comparing the in vitro activity of the construct to the in vitro activity of the antibodies or antibody fragments at an equivalent dosage in free form. The construct may be formulated by attaching the antibodies or antibody fragments to the surface of a liposome to form an antibody-liposome conjugate.

The step of measuring the therapeutic activity of the construct and the free antibodies or antibody fragments may comprise measuring one or more therapeutic activity parameters in a cell-based screening assay. For example, one or more parameters relating to cytotoxicity, cytostasis, apoptosis induction, cell morphology, cytokine production, signal transduction, immune cell activity, cellular proliferation, cellular activation and protein expression and activation may be assessed.

The invention may also include the step of selecting an antibody having enhanced activity in the multivalent construct than in free form in vitro and evaluating the effectiveness of the antibody in vivo in an animal model of a disease state, such as cancer or an autoimmune disorder.

The invention also encompasses multivalent antibody constructs formulated in accordance with the invention and their use for therapeutic purposes.

### BRIEF DESCRIPTION OF THE DRAWINGS

In drawings which describe embodiments of the invention but which should not be construed as restricting the spirit or scope of the invention in any way,

FIG. 1 is a schematic view of a synthetic scheme illustrating coupling chemistry for conjugating an antibody to the PS containing liposomes.

FIG. 2 is a graph showing Ramos cell viability, determined by MTT assay, 48 hours after treatment with free rituximab, free rituximab with secondary antibody, or rituximab bound to liposomes (at antibody-to-lipid ratios of 59 and 48 μg antibody/μmol lipid).

FIG. 3 is a graph showing Z138C cell viability, determined by MTT assay, 48 hours after treatment with free rituximab, free rituximab with secondary antibody, or rituximab bound to liposomes (at antibody-to-lipid ratios of 59 and 48 μg antibody/μmol lipid).

FIG. 4 is a table summarizing data relating to conjugation of therapeutic antibodies (trastuzumab and rituximab) to liposomes.

FIGS. 5(a)-(d) are a series of bar graphs summarizing results for in vitro treatment of cancer cell lines with free bivalent or liposomal multivalent Ab constructs. Fraction of cells affected (fa) by free or liposomal trastuzumab for (a) LCC6^{HER2} and (b) MCF7^{HER2} breast cancer cells after exposure for 5 days under various culture conditions. □, Free trastuzumab; □, Liposomal trastuzumab; HRG, heregulin. Fraction of cells affected (fa) by free or liposomal rituximab for (c) Ramos (Burkitt's lymphoma) and (d) Z-138 (mantle cell lymphoma) cell lines after exposure for 3 days in 10% serum. □, Free rituximab; □, Liposomal rituximab; ▪, Free rituximab +goat F(ab′)₂ anti-human IgG (50 μg/1 mL). Fraction of cells affected (fa) was calculated as follows: fa=1−fu, where fu was the fraction of remaining viable cells calculated as the ratio of the absorbance readings from the MTT assay for treated and untreated groups. Data represent mean ±S.E.M. from 3 independent experiments. * and ^{#} denote P<0.05 as compared to the free Ab treatment (ANOVA and Scheffe test for post-hoc comparisons).

FIGS. 6(a)-6(b) are bar graphs and Western blot analyses showing modulation of cancer cell survival pathways by multivalent liposomal Ab constructs. (a) Representative cell cycle and Western blot analyses of LCC6^{HER2} breast cancer cells after exposure to free or liposomal trastuzumab for 5 days in 10% or 0.1% serum. The concentration of trastuzumab was 200 μg/mL. (b) Representative cell cycle analysis, DAPI staining and Western blot analyses of Z-138 cells after exposure to free or liposomal rituximab for 3 days. The concentration of rituximab was 15 μg/mL. For Western blot analyses: lane 1—untreated control; lane 2—free rituximab; lane 3—liposomal rituximab; lane 4—liposome control; lane 5—liposomal irrelevant Ab; lane 6—cross-linked rituximab. For DAPI staining, yellow arrows indicate apoptotic morphology of DNA condensation or fragmentation associated with Z-138 cells exposed to various treatments. The scale bar is equivalent to 50 μm, and all images are shown at the same magnification.

FIGS. 7 (a)-(c) are fluorescence microscope digital images showing co-labeling of GM₁ glycolipid and free or liposomal form of rituximab in Z-138 mantle lymphoma cells. Free rituximab was labeled with Alexa Fluor 488, and liposomal rituximab was labeled with the fluorescent lipid DiO. Z-138 cells were incubated with fluorescently labeled free or liposomal rituximab for 18 h. After washing the cells three times with clear HBSS, CTB-Alexa Fluor 647 (2.5 μg/mL) was added to label GM₁. Cells were then washed, mounted, and imaged live. White arrows in panels a-c indicate regions that are indicative of co-localization. The scale bar in panel c is equivalent to 50 μm, and images in panel a-c are shown at the same magnification. Insets 1 and 2 show representative single and merged images of individual cells stained with free or liposomal rituximab and CTB-Alexa Fluor 647.

FIGS. 8(a) and (b) are graphs showing in vivo plasma elimination kinetics and therapeutic efficacy of free and liposome-conjugated trastuzumab in female Rag2-M mice. (a) Plasma levels of trastuzumab (∘, free trastuzumab; , liposomal trastuzumab), plasma levels of liposomal lipid (F, control liposomes without trastuzumab; ▪, trastuzumab-conjugated liposomes), and plasma trastuzumab-to-lipid ratios (Δ). The dose of trastuzumab was 6 mg/kg, and the lipid dose was 100 mg/kg in an injection volume of 0.2 mL. The initial Ab-to-lipid ratio of liposomal trastuzumab was 50 μg/μmol. Blood samples were collected and processed at various times as described in the Method section. Data represents mean ±S.E.M. from 4 mice. * denotes plasma level of liposomal trastuzumab which was statistically different from that of free trastuzumab with P<0.05 (ANOVA and Scheffe test for post-hoc comparisons). (b) In vivo efficacy of free and liposomal trastuzumab in LCC6^{HER2} breast cancer model. Trastuzumab and the irrelevant Ab, either in free or liposomal form, were given at 1 mg/kg i.v. twice weekly for 5 weeks, starting on Day 18 post inoculation of tumor cells. The lipid doses for the control liposome group were made equivalent to those of liposomal trastuzumab. Six mice were used in each group, and the data represent mean ±S.E.M.

### DETAILED DESCRIPTION OF THE INVENTION

Throughout the following description specific details are set forth in order to provide a more thorough understanding of the invention. However, the invention may be practiced without these particulars. In other instances, well known elements have not been shown or described in detail to avoid unnecessarily obscuring the present invention. Accordingly, the specification and drawings are to be regarded in an illustrative, rather than a restrictive, sense.

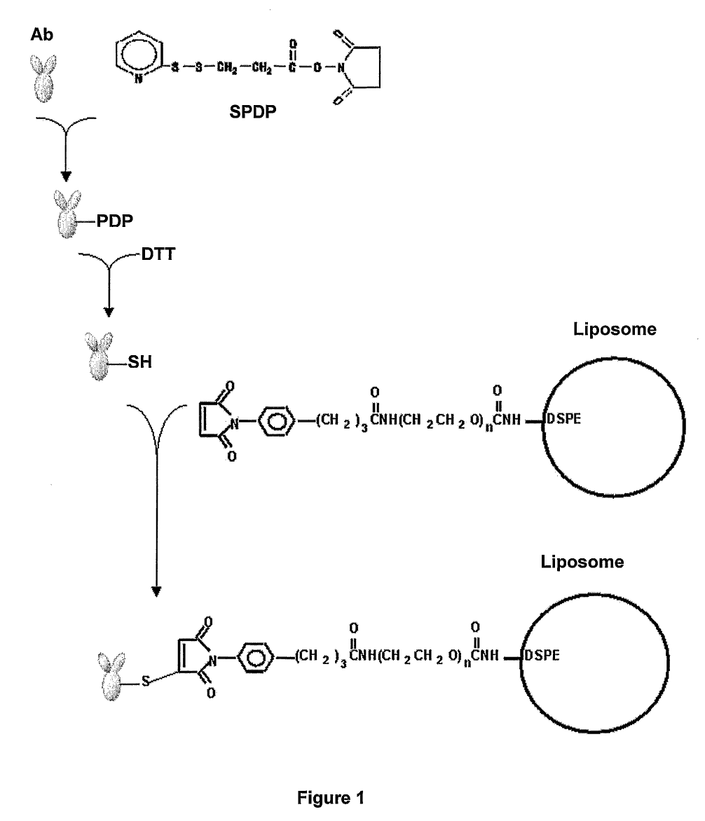

The present invention relates to a method for identifying antibodies having potential in vivo therapeutic activity by means of a cell-based in vitro assay. The invention could be employed as a rapid screening technique. As described in detail in the experimental examples below, the assay employs a multivalent antibody construct. In one preferred embodiment the construct comprises a plurality of antibodies or antibody fragments conjugated to liposomes. The antibodies or antibody fragments may be conjugated according to established procedures as shown in FIG. 1 and described further below. The plurality of antibodies may be the same or different and may be either monoclonal or polyclonal. In particular embodiments the antibodies may be human, humanized or chimeric in origin. As will be appreciated by a person skilled in the art, many different types of antibody fragments are also possible including single chain antibodies, single chain Fv fragments, and F(ab′)2, Fab, Fd and Fv fragments. As used in this patent application the term “multivalent” refers to an antibody construct, such as a liposome conjugated to multiple antibodies or antibody fragments, which has greater than two discrete antigen binding sites. The binding sites may be the same or different depending upon the antibodies or fragments employed (i.e. the binding sites may recognize the same or different antigenic epitopes).

The cell-based assay of the invention measures the therapeutic activity of the multivalent antibody construct and compares the therapeutic activity to the antibodies or antibody fragments at comparable dosage in vitro. Antibodies which show significantly increased activity in multivalent form are then selected as promising candidates for in vivo use. The efficacy of the antibodies in vivo may be tested in an animal model of a disease state in either conjugated for free form. As described further below, the disease state may be cancer or an autoimmune disorder in different embodiments of the invention.

The therapeutic activities of the antibody construct and free antibodies may be measured in vitro with reference to various cell function parameters. For example, relevant biological parameters may include cytotoxicity, cytostasis, apoptosis induction, cell morphology, cytokine production, signal transduction, cell proliferation and activation status and protein expression and activation. Other biologically appropriate endpoints may also be considered. In some particular embodiments of the invention therapeutic activity may be assessed by means of a MTT assay or XTT assay (cell proliferation); a trypan blue exclusion assay (cell viability); membrane phosphoserine asymmetry, annexin 5 binding and caspase assays (apoptosis); and cytokine production (cell function).

The assay may employ a cell line expressing an antigen specific to the antibodies or antibody fragments under consideration. For example, the cell line may be a cancer cell line and the antigen may be a tumour cell antigen. Examples of cell lines described in the following examples include leukemic cell lines such as Ramos and Z138C and breast cancer cell lines such as LCC6^{HER2} and MCF7^{HER2}. The cell type may be of primary origin from a patient or animal or it may be a cell line that is biologically relevant or models an aspect of the disease to be treated.

As is well known in the art, liposomes are well-suited for encapsulating therapeutic agents such as chemotherapeutic drugs for targeted drug delivery. In one embodiment of the invention therapeutic agents separate from the antibodies or antibody fragments may be tested in vitro or in vivo in combination with the antibody constructs. Such tests can be used to identify drugs that augment or synergistically enhance a desired therapeutic effect. The therapeutic agents can be small molecules, peptides, nucleic acids or antibodies known to be effective in the treatment of disease, such as cancer. While co-delivery of antibodies and chemotherapeutics is not new, in most prior art approaches the antibodies are employed to bind to a target antigen or antigen and the therapeutic effect is achieved by the encapsulated drug. In the present invention antibody conjugated liposomes are shown to have an enhanced therapeutic effect, even in the absence of a co-delivered chemotherapeutic agent.

As described further below, it is believed that the multivalent liposomal form of some antibodies may demonstrate increased therapeutic activity in comparison to the same antibodies in free, bivalent form due to modulation of malignant cell survival signally pathways via extension cross-linking of the antibody/target antigen complex. In order to achieve this result, the number of antibodies conjugated to each liposome and the lipid make-up of the liposome may be important. In some embodiments of the present invention, the number of antibodies per liposome is within the range of about 20 to 50 and the antibody/lipid ratio is within the range of about 40-75 μg/μmol. The ratios and selection of lipids may vary without departing from the invention. For example the liposome may consist of lipids including phosphoglycerides and sphingolipids, representative examples of which include phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine or dilinoleoylphosphatidylcholine. Other compounds lacking in phosphorus, such as sphingolipid and glycosphingolipid families are also contemplated. Additionally, the amphipathic lipids described above may be mixed with other lipids including triacyglycerols and sterols. In one particular example the liposome may consist of lipids in the following ratios: DSPC 49%, Cholesterol 45%, DSPE-PEG 2000 5%, DSPE-PEG 2000-maleimide 1%.

The invention also encompasses multivalent antibody constructs identified as having potential in vivo activity. Particular constructs are identified below which are shown to have enhanced activity in vitro and in vivo in comparison to the same antibodies administered in free, bivalent form. Such compositions have utility as pharmaceutical compositions. As will be appreciated to a person skilled in the art, pharmaceutical compositions containing antibodies may be administered by various means including intraarticularly, intravenously, subcutaneously, or intramuscularly.

The invention also relates to use of multivalent antibody constructs for treatment of disease states such as cancer or autoimmune disorders. In one embodiment the disease states may be of the hemopoetic system such as lymphomas and leukaemias. In other embodiments, the invention provides a method of identifying therapeutically active antibodies for treating solid tumours and the metastasis of such tumours such as tumours of the lung or breast.

The following examples will further illustrate the invention in greater detail although it will be appreciated that the invention is not limited to the specific examples.

### EXAMPLE 1.0

### 1.1 Materials and Methods

### 1.1.1 Liposome Formation

Large unilamellar vesicles (LUVs) were prepared by the extrusion method (DSPC 49%, Cholesterol 45%, DSPE-PEG 2000 5%, DSPE-PEG 2000-maleimide 1%). Briefly, lipids were dissolved in chloroform at the required molar ratio, labeled with the non-exchangeable, non-metabolizable lipid marker, 3H-CHE and dried to a thin film under a stream of nitrogen gas. Subsequently, the lipid was placed in a high vacuum for 3 h to remove any residual solvent. The lipid films were then hydrated at 65° C. by mixing with the appropriate buffer (300 mM CuSO4, 300 mM CoSO4, 300 mM ZnSO4 and 300 mM MnSO4). The mixture was subjected to five cycles of freeze-and-thaw (5 minutes each, freezing in liquid nitrogen and thawing at 65° C.). The formed multilamellar vesicles (MLV's) were extruded 10 times through stacked polycarbonate filters of 0.08 μm and 0.1 μm pore size at 65° C. (Extruder, Northern lipids). The resultant LUVs typically possessed mean vesicular diameters in the range 110 nm±10 nm as determined by quasi-elastic light scattering using the Nicomp submicron particle sizer model 370/270. The LUVs' external buffer was exchanged with a buffer at pH 7.5 (300 mM sucrose, 20 mM HEPES, 15 mM EDTA) using sephadex G-50 size exclusion chromatography.

### 1.1.2 Thiolation and Conjugation of Antibodies to Liposomes

The antibody is conjugated to the liposomes according to established procedures as shown in FIG. 1 and described in detail in Section 2.1.2 below.

Briefly, the antibody Ab was first modified with SPDP which reacts with the primary amines on the Ab. The free thiol group on the Ab was generated after DTT reduction, and would react with the maleimide group on the distal end of the PEG linked to DSPE. The reactive lipid for the conjugation was DSPE-PEG 2000-MAL, and the amount was 1 mol % in the liposomes.

### 1.2 Results

### 1.2.1 In Vitro B Lymphoma Cell Viability Post Rituximab Treatment Determined by MTT Assay.

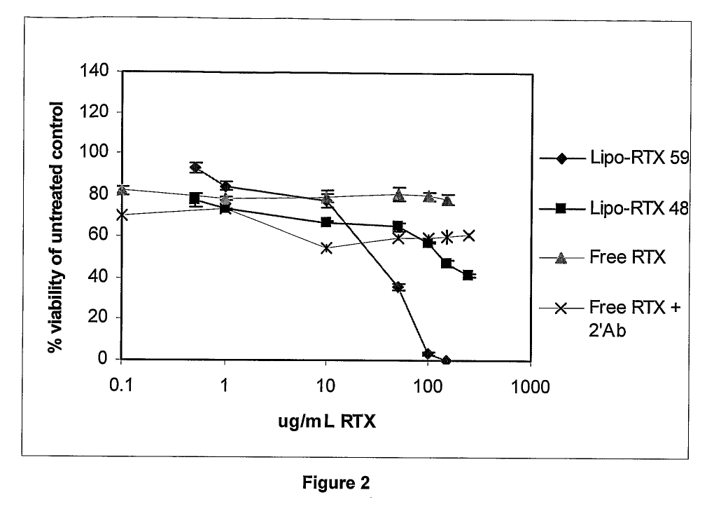

As shown in FIG. 2, Ramos cells were treated with free rituximab (Free RTX), free rituximab with secondary antibody (Free RTX+2″Ab), or Rituximab bound to liposomes at antibody-to-lipid ratios of 59 and 48 μg antibody/μmol lipid (Lipo-RTX 59 and Lipo-RTX 48 respectively) for 48 hours and then the viability of the cells was determined by the MTT assay.

Rituximab bound to liposomes at antibody-to-lipid ratios of 59 and 48 μg antibody/μmol lipid (Lipo-RTX 59 and Lipo-RTX 48 respectively) showed superior potency to free Rituximab in the presence or absence of secondary antibody (FIG. 2). Liposome alone (5 mM) showed 95+10% cell viability whereas 2′ cross-linking Ab alone (50 ug/mL) showed 82+8.2% cell viability.

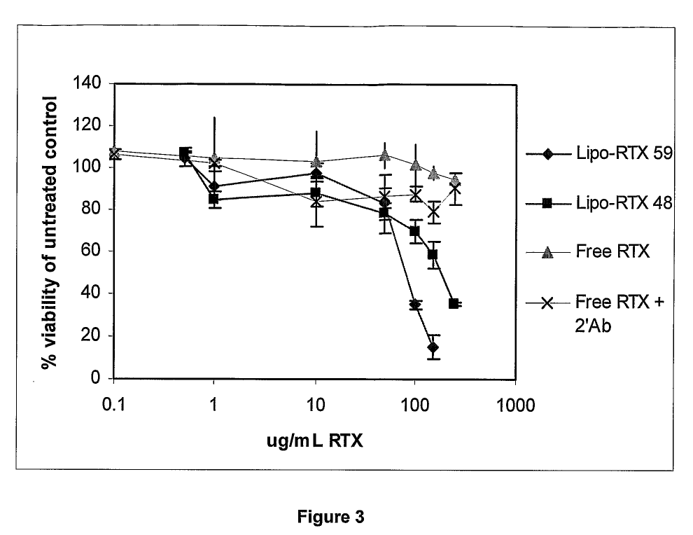

As shown in FIG. 3, Z138C cells were treated with free rituximab (Free RTX), free rituximab with secondary antibody (Free RTX+2″Ab), or Rituximab bound to liposomes at antibody-to-lipid ratios of 59 and 48 μg antibody/μmol lipid (Lipo-RTX 59 and Lipo-RTX 48 respectively) for 48 hours and then the viability of the cells was determined by the MTT assay

Rituximab bound to liposomes (Lipo-RTX 59 and Lipo-RTX 48) showed superior potency to free Rituximab in the presence or absence of secondary antibody (FIG. 3). Liposome alone (5 mM) showed 105+9.2% cell viability whereas 2′ cross-linking Ab alone (50 ug/mL) showed 80+7.3% cell viability.

This example demonstrates that free rituximab in vitro has minimal or no cytotoxic activity toward the B lymphoma cell lines (Ramos and Z138C). By contrast, an anti CD20 monoclonal antibody, rituximab, when coupled to a liposome composed of DSPC 49%, Cholesterol 45%, DSPE-PEG 2000 5%, DSPE-PEG 2000-maleimide 1% had greatly enhanced activity of the antibody. As explained above, the enhanced activity may possibly be through a mechanism of hyper-cross-linking the CD20 antigen.

This example exemplifies the inventive methodology for the identification of therapeutically active antibodies comprising (a) attaching antibodies to liposomes; (b) evaluating in vitro the therapeutic activity of the liposome antibody conjugate; and (c) selection of antibodies that exhibit enhanced therapeutic activity when added as a liposome conjugated when compared to added as free antibody.

### EXAMPLE 2.0

### 2.1 Materials and Methods

### 2.1.1 Cell Lines

Cell lines. MCF-7^{HER2} cells were a kind gift from Dr. Moulay Alaoui-Jamali (McGill University, Montreal, QC, Canada). MDA-MB-435/LCC6 were generously provided by Dr. Robert Clarke (Georgetown University, Washington, D.C.), and LCC6^{HER2} cells were transfected with the human expression plasmid pREP9 containing the full-length human c-erbB-2 cDNA (provided by Dr. Ming Tan, M.D. Anderson Cancer Center, Houston, Tex.) as previously described ²⁸. MCF-7^{HER2} and LCC6^{HER2} cells were grown in DMEM (Stem Cell Technologies, Vancouver, BC, Canada), supplemented with 2 mM L-glutamine, 10% fetal bovine serum (FBS; Hyclone, Logan, Utah) and 500 μg/mL Geneticin (LCC6^{HER-2}) or 100 μg/mL Geneticin (MCF-7^{HER-2}). For all experiments, MCF-7^{HER2} and LCC6^{HER2} cells were cultured without Geneticin for 1 week before the experiment. Ramos (RA 1) cell line was purchased from ATCC (Manassas, Va.), and maintained in culture conditions according to ATCC instructions. Z-138 cell line was generously provided by Dr. Zeev Estrov (University of Texas), and maintained in RPMI1640 medium (Stem Cell Technologies) supplemented with 2 mM L-glutamine, 10% FBS, and 1% penicillin/streptomycin. All cells were maintained at 37° C. in a humidified atmosphere containing 5% CO₂.

### 2.1.2 Preparation of Liposome-Conjugated Ab

Liposomes, composed of DSPC/CHOL/DSPE-PEG₂₀₀₀/DSPE-PEG₂₀₀₀-MAL, were prepared by the extrusion procedure ³⁹. The resulting mean vesicle diameter was 100-120 nm as determined by quasi-elastic light scattering using the Nicomp submicron particle sizer model 370/270. Therapeutic Ab was conjugated to liposomes according to an established procedure ¹⁶. Briefly, SPDP (Pierce) was dissolved in ethanol, and 80 μL of this solution (12.5 mM) was diluted with 920 μL HBS to give a final concentration of 1 nmol/μL. The Ab (8-9 mg) was reacted with SPDP in a 1:5 Ab to SPDP mole ratio for 25 min at room temperature, and was subsequently passed down a Sephadex G-50 column equilibrated with sodium acetate buffer (100 mM sodium acetate/50 mM NaCl, pH 4.5). Fractions containing the Ab with absorbance at 280 nm greater than 1 were pooled, and were added to DTT powder (Pierce) to give a concentration of 25 mM DTT. This mixture was incubated at room temperature for 25 min. The thiolated Ab was then isolated by size exclusion chromatography with a Sephadex G-50 column equilibrated with HBS, and was immediately added to liposomes (10 mM final concentration). The mixture was incubated at room temperature for 18 h with gentle stirring. At the end of the reaction, the mixture was passed down a Sepharose CL-4B column equilibrated with HBS to separate the free Ab from the liposome-conjugated Ab. Liposomes were labeled with [³H]CHE to facilitate the determination of liposomal lipid concentration by liquid scintillation counting (Packard scintillation counter model 1900 TR) with aliquots mixed with 5.0 mL Pico-fluor 15 scintillation fluid (Packard Biosciences, The Netherlands). The amount of Ab conjugated to liposomes was determined using the Pierce Micro BCA protein assay kit in the presence of 0.5% Triton X-100.

### 2.1.3 Cell Viability Assay.

Cells (3,500 cells/well for LCC6^{HER2} and MCF7^{HER2}, and 20-50,000 cells/well for Ramos and 20,000 cells/well for Z-138) were seeded in 96-well plates. In the case of LCC6^{HER2} and MCF7^{HER2} cells, treatment was added after an overnight incubation to allow cell adherence to plates. In the case of Ramos and Z-138 cells, treatment was added on the same day after seeding. Cell viability was determined using 3-(4,5-dimethylthiazol-2-yl)-2,5-diphenyltetrazolium bromide (MTT) as previously described ²⁸ or Alamar Blue added in 10 μL/well with an 18-h incubation before reading fluorescence in a Fluorstar (v 1.2) plate reader (BMG Labtechnologies, Offenburg, Germany).

### 2.1.4 Flow Cytometric Analyses and DAPI Staining.

Cells were harvested after treatment and fixed in cold 70% ethanol adjusted to 1×10⁶ cells/mL and 2×10⁶ cells/mL for cell cycle analyses and DAPI staining, respectively. In the case of cell cycle analyses, cells were stained in propidium iodide (PI) buffer [50 μg/mL PI, 1 mg/mL RNase A (Sigma), and 0.1% Triton X-100 in PBS] at 37° C. for 15 min as previously described ²⁸. Cells were then chilled on ice for 1 h and analyzed on a FACSCalibur flow cytometer (Becton Dickinson, San Jose, Calif.) with 20,000 events collected for each sample. In the case of DAPI staining, cells were incubated in DAPI staining solution (0.1 μg/mL DAPI, 1 mg/mL RNase A and 0.1% Triton X-100 in PBS) for 15 min, followed by chilling on ice for 30 min. Cells were cytospun onto a glass slide, and viewed with a Leica DMLB fluorescence microscope. Images were captured with a Retiga 1300i digital camera and analyzed by the computer software OpenLab 3.5.1 (Improvision).

### 2.1.5 Western Immunoblot Analyses.

The following antibodies were used in this study: anti-p-HER2-Tyr-1248/Tyr-1173; anti-HER2; anti-p-Akt-Ser-473 and anti-Akt (all were rabbit polyclonal, New England Biolabs) and anti-human β-actin (mouse monoclonal, Sigma). The secondary antibody was horseradish peroxidase-conjugated anti-mouse or anti-rabbit IgG (Promega, Madison, Wis.). Proteins were detected by enhanced chemiluminescence (Amersham Pharmacia Biotech, Buckinghamshire, England), and visualized after exposure to Kodak autoradiography film. Equivalent amounts of protein (30 μg/lane determined by Bradford assay) were resolved by 12% SDS-polyacrylamide pre-made gels (Bio-Rad).

### 2.1.6 Co-Labeling of GM₁ and Free or Liposomal Rituximab in Z-138 Cells

Cells (4×10⁶/mL) were seeded in 24-well plates and incubated for 18 h with the following: free rituximab, liposome-conjugated rituximab, liposome-conjugated irrelevant Ab, and free rituximab +goat F(ab′)₂ anti-human IgG (50 μg/mL). Free rituximab was labeled with Alexa Fluor 488 according to the instructions of the manufacturer's labeling kit (Molecular Probes). Liposomes conjugated with rituximab or irrelevant Ab were labeled with the fluorescent lipid tracer DiO (Molecular Probes). At the end of incubation, cells were washed in cold medium, and were re-suspended in 2.5 μg/mL cholera toxin subunit B (CTB) labeled with Alexa Fluor 647 in cold medium for 10 min on ice. Cells were washed and mounted in chilled HBSS onto a glass slide, and were imaged live with the same fluorescence microscope as mentioned above. Images were captured and analyzed with the same digital camera and software as described above.

### 2.1.7 Animal Studies.

All in vivo studies were completed using protocols approved by the University of British Columbia's Animal Care Committee, and were in accordance with the current guidelines of the Canadian Council of Animal Care. Female Rag2-M mice (20-22 g) were obtained from Taconic. For plasma elimination studies, LCC6^{HER2} tumor bearing mice were injected with 6 mg/kg (in 200 μL) free or liposomal trastuzumab. At various times post injection, blood was collected by cardiac puncture and placed into EDTA-coated microtainer tubes. Plasma was isolated from blood samples by centrifugation at 1000×g for 15 min. Aliquots of the plasma were used to determine trastuzumab levels by a colorimetric ELISA assay. Briefly, a rabbit anti-human IgG Fc fragment (MP Biomedicals, Irvine, Calif.) was used for coating and capturing trastuzumab. A horseradish peroxidase-conjugated rabbit anti-human whole IgG (Sigma) was used for detection, with orthophenylenediamine (Sigma) added as substrate. To determine the levels of trastuzumab in plasma, absorbance at 405 nm was measured and compared to values from a standard curve constructed from known amounts of trastuzumab. Plasma liposomal lipid levels were determined by liquid scintillation counting of [³H]CHE radioactivity. For in vivo efficacy studies, mice were inoculated s.c. unilaterally with 5×10⁶ LCC6^{HER2} cells. On Day 18 post-inoculation of tumor cells, mice were treated with free or liposomal trastuzumab twice a week with a dose of 1 mg/kg for 5 weeks. Tumor measurements were made twice a week with a caliper.

### 2.1.8 Statistical Analyses.

All data values are reported as mean ±standard error of the mean (S.E.M.). Two-way analysis of variance (ANOVA) was performed to determine statistical significant differences of the means, with Scheffe test used for post-hoc multiple comparisons. An alpha value of 0.05 was chosen, and all tests were two-tailed.

### 2.2 Results

### 2.2.1 Conjugation of Therapeutic Ab to DSPC/CHOL/DSPE-PEG₂₀₀₀ Liposomes.

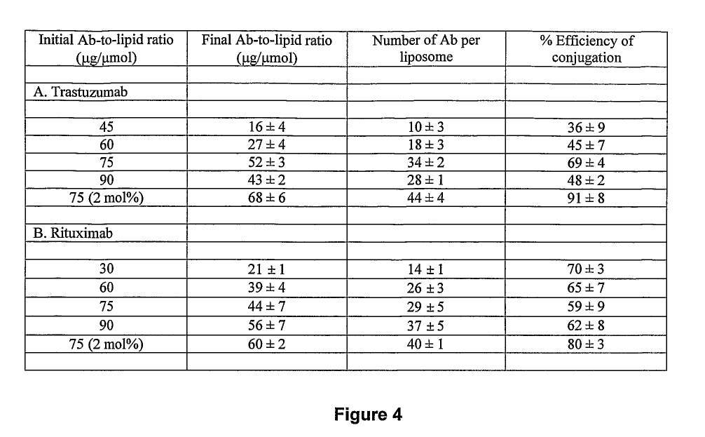

Trastuzumab and rituximab were conjugated to DSPC/CHOL/DSPE-PEG₂₀₀₀ liposomes at various initial Ab-to-liposomal lipid ratios (FIG. 4). The lipid composition of the liposomes was DSPC/CHOL/DSPE-PEG₂₀₀₀/DSPE-PEG₂₀₀₀-MAL 49:45:5:1 (mole ratio), and in some cases, the amount of DSPE-PEG₂₀₀₀-MAL was 2 mol % as specified in parentheses in the table of FIG. 4. Both antibodies were first modified with the bifunctional cross-linker SPDP in order to conjugate to the liposome containing a maleimide (MAL) group on the PEG terminus of DSPE-PEG₂₀₀₀ through a thioether linkage ¹⁶. Such modification did not compromise the binding of the SPDP-modified Ab to target cells when compared to the unmodified Ab, as determined by flow cytometry (data not shown). The final Ab-to-lipid ratios were determined from 3-6 independent experiments. With an initial Ab-to-lipid ratio of 75 μg/μmol, a final ratio of 52±3 μg/μmol or 44±7 μg/μmol was obtained with 1 mol % DSPE-PEG₂₀₀₀-MAL for trastuzumab or rituximab, respectively. If the level of the reactive lipid was increased to 2 mol % DSPE-PEG₂₀₀₀-MAL, a final ratio of 68±6 μg/μmol or 60±2 μg/μmol was obtained for trastuzumab or rituximab, respectively. This represents a range of conjugation efficiencies of 80-91%. The % efficiency of conjugation was calculated by the following equation: % efficiency=Final Ab-to-lipid ratio/Initial Ab-to-lipid ratio×100%. For subsequent in vitro and in vivo functional assessments, the liposomal Ab constructs had similar Ab-to-lipid ratios in the range of 50-70 μg/μmol, which is equivalent to approximately 38-45 copies of Ab present in a single liposome. The number of Ab per liposome was estimated from the final Ab-to-lipid ratios assuming that one μmol of 100-nm liposomes contains 6.23×10¹² vesicles ¹⁷ and the molecular weights of trastuzumab and rituximab are 148,220 and 145,000, respectively.

### 2.2.2 Reduction in Cell Viability After Exposure to Multivalent Liposomal Ab Constructs.

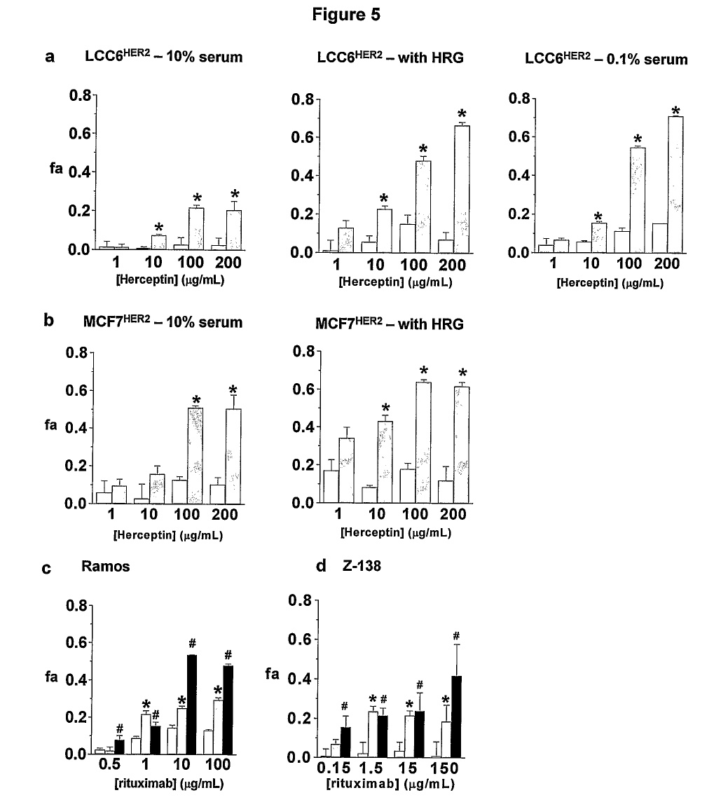

To test if the biological activities of the Ab would be enhanced when presented in the multivalent format, the activity of the free, bivalent Ab was compared to that of the multivalent construct in the respective cancer cell lines. Standard cell viability assays were used to determine the fraction of cells affected (fa) upon treatment. For trastuzumab, testing was conducted in LCC6^{HER2} and MCF7^{HER2} breast cancer cell lines under various culture conditions (FIG. 5a, b). The fa value for free trastuzumab tested in the presence of 10% serum for 5 days was at most 0.12, and no improvement in the activity was observed even when tested at 1 mg/mL (data not shown). In contrast, the fa values of the multivalent liposomal trastuzumab were significantly higher in LCC6^{HER2} (above 10 μg/mL) and MCF7^{HER2} cells (above 100 μg/mL). Consistent with previous observations ¹⁸, the activity of free trastuzumab was slightly increased when tested in the presence of 0.1% serum or heregulin (the binding ligand associated with HER2/HER3 dimerization), with the fa value increased to a maximum of 0.2. The activity of multivalent liposomal trastuzumab in the presence of 0.1% serum or heregulin was further enhanced, with fa values approaching 0.7 when tested at 200 μg/mL in both LCC6^{HER2} and MCF7^{HER2} cells.

For rituximab, testing was conducted in a Burkitt's lymphoma cell line, Ramos (FIG. 5c), and a mantle cell lymphoma cell line, Z-138 (FIG. 5d). Free rituximab cross-linked by an anti-human IgG F(ab)′ was included as a control ⁷. Similar to liposomal trastuzumab, the multivalent liposomal rituximab was significantly more effective than the free, bivalent form for the range of Ab concentrations tested in the two lymphoma cell lines. Cross-linked rituximab was more effective than liposomal rituximab when tested at Ab concentrations above 10 μg/mL in Ramos cells (P<0.05), whereas the activity of liposomal rituximab was not significantly different from that of cross-linked rituximab in Z-138 cells (P>0.05). In all of the cell lines evaluated here, plain liposomes, liposomal irrelevant Ab construct and the cross-linking F(ab)′ alone exhibited minimal activity, with fa values <0.03 (data not shown).

### 2.2.3 Modulation of Cell Survival Pathways by Multivalent Liposomal Ab Constructs.

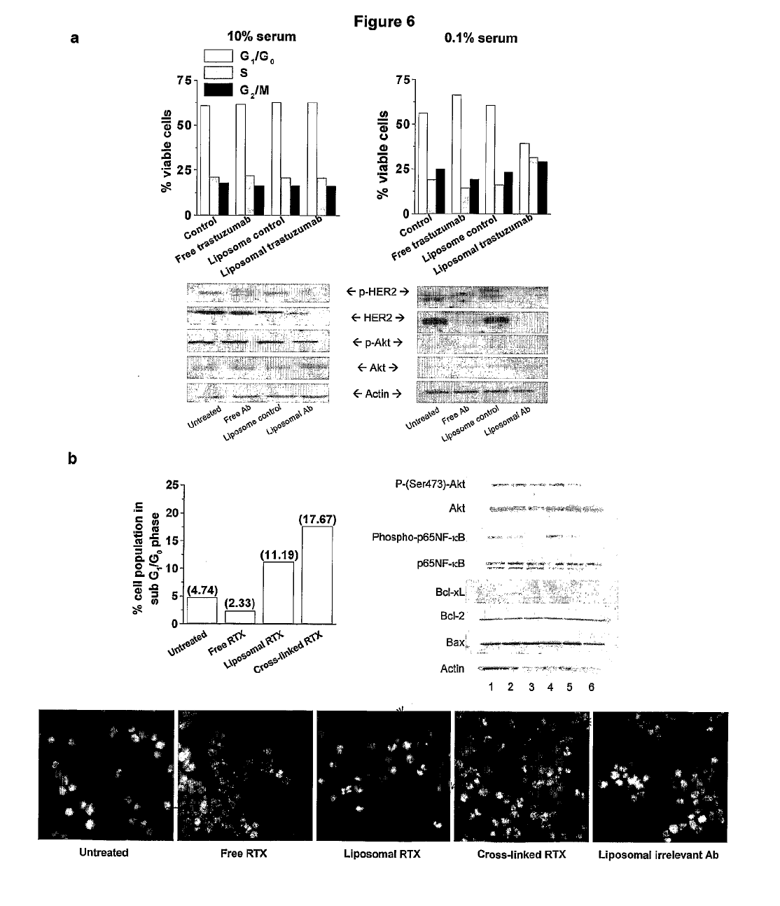

To understand the enhancement in biological activity effected by multivalent liposomal Ab constructs, molecular characterizations of the Ab-mediated changes in cell survival pathways were performed. Testing of trastuzumab was conducted in LCC6^{HER2} cells, and two important observations can be made (FIG. 6a). First, consistent with previous reports that trastuzumab activity could be increased when cells were treated in reduced serum levels ¹⁸, changes in the cell cycle status and in the expression levels of HER2 and its downstream target Akt engendered by both free and liposomal trastuzumab were more prominent when cells were treated in 0.1% serum. Secondly, while free trastuzumab was capable of inducing a small increase in the percentage of cells in G₁/G₀ phase and down-regulation of p-HER2 and total HER2 in cells treated under reduced serum levels, it was not capable of reducing p-Akt level. In contrast, liposomal trastuzumab induced a complete loss of p-Akt, p-HER2 and total HER2, with the cell cycle profile showing a substantial decrease in the percentage of cells in G₁/G₀ phase with a concomitant increase in S phase. This demonstrates the difference in the ability of free and liposomal trastuzumab in modulating the Akt-mediated cell signaling pathway which is important to cancer cell proliferation, survival and chemoresistance ¹⁹⁻²¹.

In the case of rituximab, testing was conducted in Z-138 cells (FIG. 6b). Results from cell cycle analyses, DAPI staining, and western analyses of selected cell survival molecules demonstrated that cells treated with free rituximab were not different from untreated cells. Only through cross-linking or liposome conjugation was the activity of rituximab increased, with more cells undergoing apoptosis as reflected by the morphological changes and the increases in the percentage of cells in sub-G₁/G₀ phase for liposomal and cross-linked rituximab. Both liposomal (FIG. 6b, Western blot, lane 3) and cross-linked rituximab (FIG. 6b, Western blot, lane 6) reduced the expression levels of phosphorylated p65NF-κB and the downstream Bcl-xL without changing the Bcl-2 to Bax ratio. Interestingly, cross-linked rituximab was also capable of reducing the expression level of p-Akt, which has been implicated as an upstream regulator of NF-κB ²²⁻²⁴. A 2-fold increase in caspase 3/7 activity was observed in cells treated with cross-linked rituximab but not with liposomal rituximab (data not shown). Further investigations to discern the molecular effects of liposomal rituximab from those of cross-linked rituximab are warranted.

### 2.2.4 Co-Labeling of GM₁ and Free or Liposomal Form of Rituximab in CD20⁺ Cells

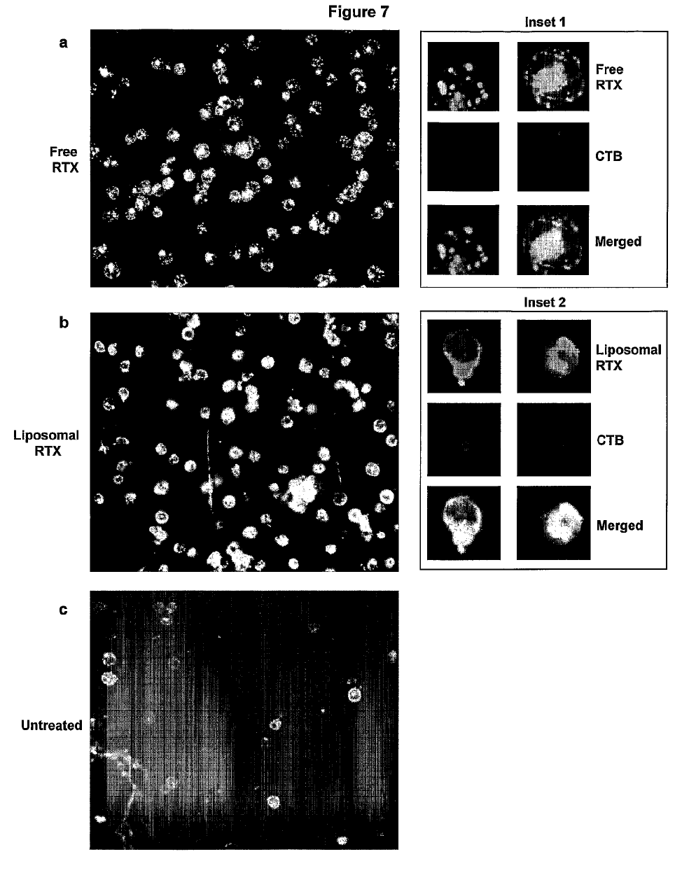

Hyper-cross-linking of CD20/rituximab is thought to involve membrane rafts ⁵. To determine if GM₁, a glycolipid that partitions into membrane rafts, was co-localized with CD20, Z-138 cells were stained sequentially with fluorescently labeled free or liposomal rituximab and CTB-Alexa Fluor 647. Consistent with a recent study which reported the constitutive association of CD20 with rafts ²⁵, the cell surface of Z-138 cells stained with free rituximab generally appeared punctate (FIG. 7a). In contrast, cells stained with liposomal rituximab appeared patchy with regions enriched in both fluorescence signals on the cell surface (FIG. 7b). Although regions indicative of co-localization of GM₁ and free rituximab were found in the images, more regions of co-localization of GM₁ and liposomal rituximab were identified. Taken together, these results suggest that CD20-associated rafts may coalesce to form bigger domains on the cell surface upon multivalent interactions with liposomal rituximab.

### 2.2.5 Plasma Elimination Kinetics and Anti-Tumor Efficacy of Liposomal Trastuzumab in a Breast Tumor Xenograft

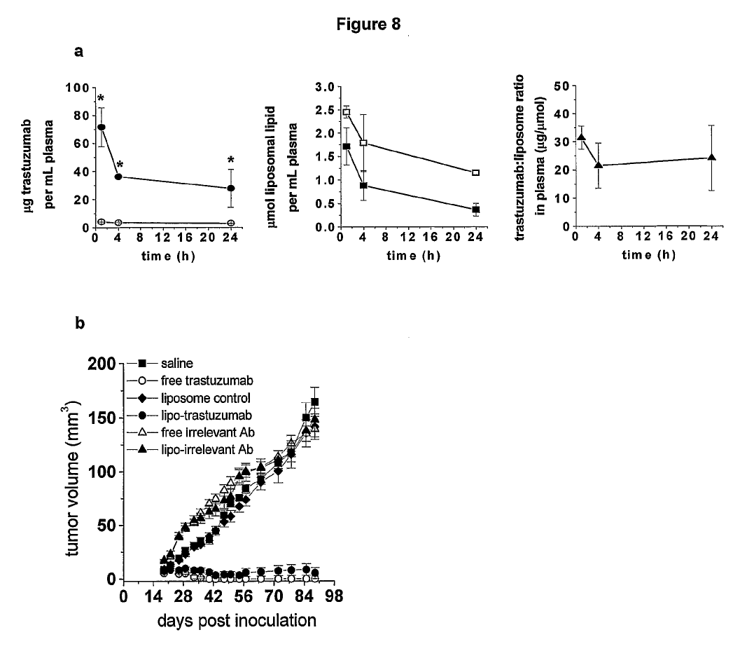

Plasma elimination of trastuzumab in free or liposomal form was evaluated in female Rag2-M mice (FIG. 8a), and two important observations can be made. First, conjugation of trastuzumab to liposomes significantly increased the plasma levels of the Ab, compared to the free form (P=0.00001, 0.01 and 0.04 for 1, 4 and 24 h, respectively). Secondly, conjugation of trastuzumab did not significantly alter the circulation longevity of the liposome carrier (P=0.3, 0.2 and 0.2 for 1, 4 and 24 h, respectively). These two attributes are pertinent to the development of liposome carriers that are intended for the co-delivery of Ab and chemotherapeutics as prolonged circulation longevity of both Ab and chemotherapeutics via liposomal delivery is believed to increase tumor exposure and thus therapeutic activity.

The anti-tumor activity of trastuzumab in free or liposomal form was evaluated in the LCC6^{HER2} breast tumor xenograft model (FIG. 8b). Since estrogen receptor-negative (ER⁻) breast cancer is more aggressive and poor in prognosis 6, the ER⁻LCC6^{HER2} model was chosen in this study. Although free trastuzumab exhibited minimal in vitro activity, it was very effective in vivo, with long-term tumor growth inhibition achieved in the treated animals. This observation supports the notion that the in vivo activity of trastuzumab depends on the presence of Fc_{γ}R-expressing effector cells such as macrophages and natural killer cells ²⁷. Similarly, long-term tumor growth inhibition was observed in mice treated with multivalent liposomal trastuzumab, indicating that anti-tumor activity was not compromised by conjugating trastuzumab to liposomes. Tumor cell viability post treatment in vivo was comparable for free and liposomal trastuzumab, with 15% and 23% for free and liposomal trastuzumab, respectively, as determined according to an established protocol ²⁸.

As discussed above, multimeric assembly or clustering of receptors on cell surface has emerged as a common theme to a wide variety of biological processes. In malignant cells, various cellular signals including induction of apoptosis, inhibition of cell growth/survival, or internalization of surface molecules could be triggered or enhanced upon extensive cross-linking with multimeric ligand or antibodies (and fragments), suggesting that receptor or antigen clustering via multivalent interactions can modulate cell survival ⁷⁻¹⁰. In light of these observations, the inventors hypothesized that multivalent antibody constructs generated by the grafting of antibody molecules onto liposome membrane surfaces would modulate malignant cell survival signaling pathways via extensive cross-linking of target/Ab complex.

Several lines of evidence from this study support this hypothesis. First, when rituximab was presented in the multivalent liposomal form, it was capable of inducing regions of enriched GM₁ staining on the cell surface as compared to the free, bivalent form (FIG. 7). Previous biophysical analyses of the interactions of biotinylated liposomes with another surface containing neutravidin or avidin demonstrated that the liposomes could deform and spread without rupture upon binding ²⁹, and that multiple biotin-avidin-biotin cross-bridges were concentrated in the “contact zone” between the liposome surface and the interacting surface ³⁰. The enrichment in GM₁ staining when cells were exposed to liposomal rituximab is thus in line with these previous observations, and is possibly due to an increase in membrane raft size mediated through the multivalent liposomal rituximab/CD20 cross-linking.

Secondly, using trastuzumab and rituximab as examples, significant increases in the in vitro antibody activity were demonstrated in the respective cancer cell lines when the antibodies were presented in the multivalent liposomal form as compared to the free, bivalent form (FIG. 5). The free, bivalent antibodies were only active when tested in the animal models, which may be explained by the involvement of Fc_{δ}R-expressing effector cells such as macrophage and natural killer cells that are thought to mediate the cross-linking of target/Ab complex ^{7, 31}. As indicated in this example, the inventors' results demonstrate that key cell survival signaling molecules were down-regulated when the cancer cells were exposed to the multivalent liposomal antibodies. In the case of trastuzumab, a difference in the ability of free and liposomal form of the Ab to reduce the expression levels of the target molecule HER2 and the downstream molecule Akt was observed. The PI3K-Akt signaling pathway has been demonstrated to play a key role in cancer cell proliferation, survival and chemoresistance in breast and other cancers, and represents a pathway that can be exploited for the development of novel therapeutics ¹⁹⁻²¹. Although free trastuzumab was capable of down-regulating the active, phosphorylated form of HER2, it did not have an effect on the expression of the downstream molecule Akt and its active phosphorylated form. In contrast, multivalent liposomal trastuzumab was capable of down-regulating the active, phosphorylated form of Akt in addition to the target receptor HER2 (FIG. 6). This finding agrees well with the in vitro cell viability results, and suggests the importance of multivalent interactions of trastuzumab with its target molecule. In the case of rituximab, a similar trend was observed, where only upon presenting the Ab in the multivalent form via either liposome conjugation or secondary F(ab)′ cross-linking was the Ab activity significantly increased over that of the free, bivalent form. Importantly, multivalent liposomal rituximab was capable of down-regulating the phosphorylated form of NF-κB, which is a transcription factor that has been suggested to play a pivotal role in the regulation of cell proliferation and survival in lymphomas including the aggressive mantle cell lymphoma ³², as well as the downstream, anti-apoptotic Bcl-xL which has been shown to protect cells from drug cytotoxicity ³³⁻³⁵.

Considering that therapeutic Ab such as trastuzumab and rituximab are frequently combined with chemotherapy in treating various forms of cancer in the clinic ^{36,37}, developing liposome formulations of therapeutic Ab would offer the following advantages. First, from a molecular perspective, the multivalent liposomal Ab constructs which were able to down-regulate pivotal signaling molecules that mediate chemoresistance can potentially sensitize the cancer cells to drug-induced cytotoxicity, thus improving the responses to chemotherapy. The use of the multivalent Ab constructs can also provide an effective means of screening Ab/drug combinations, as it would overcome the problem of inaccurate assessment due to the use of an inactive form of Ab. Secondly, from a pharmaceutical and clinical perspective, liposomes are well suited for the development of a delivery system to co-deliver Ab and chemotherapeutics, since liposomal delivery has emerged as a mature and versatile technology that can be applied to a wide variety of medicinal agents ³⁸. The inventors' in vivo results demonstrate that two important formulation attributes for the development of liposomes intended for the co-delivery of Ab/drug combinations could be achieved, including 1) significant increases in the plasma Ab levels by liposomal delivery, and 2) prolonged circulation longevity of the liposome carrier. The present invention therefore enables screening of Ab/drug combinations for optimal therapeutic effects and delivery of such combinations via liposome technology to improve cancer therapy.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

### REFERENCES

1. Carter, P. Improving the efficacy of antibody-based cancer therapies. Nat Rev Cancer 1, 118-129 (2001).
2. Presta, L. G., Shields, R. L., Namenuk, A. K., Hong, K. & Meng, Y. G. Engineering therapeutic antibodies for improved function. Biochem Soc Trans 30, 487-490 (2002).
3. Groner, B., Hartmann, C. & Wels, W. Therapeutic antibodies. Curr Mol Med 4, 539-547 (2004).
4. Nagy, P., Jenei, A., Damjanovich, S., Jovin, T. M. & Szolosi, J. Complexity of signal transduction mediated by ErbB2: clues to the potential of receptor-targeted cancer therapy. Pathol Oncol Res 5, 255-271 (1999).
5. Deans, J. P., Li, H. & Polyak, M. J. CD20-mediated apoptosis: signalling through lipid rafts. Immunology 107, 176-182 (2002).
6. Todorovska, A. et al. Design and application of diabodies, triabodies and tetrabodies for cancer targeting. J Immunol Methods 248, 47-66 (2001).
7. Shan, D., Ledbetter, J. A. & Press, O. W. Apoptosis of malignant human B cells by ligation of CD20 with monoclonal antibodies. Blood 91, 1644-1652 (1998).
8. Ghetie, M. A., Bright, H. & Vitetta, E. S. Homodimers but not monomers of Rituxan (chimeric anti-CD20) induce apoptosis in human B-lymphoma cells and synergize with a chemotherapeutic agent and an immunotoxin. Blood 97, 1392-1398 (2001).
9. Hommelgaard, A. M., Lerdrup, M. & van Deurs, B. Association with membrane protrusions makes ErbB2 an internalization-resistant receptor. Mol Biol Cell 15, 1557-1567 (2004).
10. Miller, K. et al. Design, construction, and in vitro analyses of multivalent antibodies. J Immunol 170, 4854-4861 (2003).
11. Wittel, U. A. et al. The in vivo characteristics of genetically engineered divalent and tetravalent single-chain antibody constructs. Nucl Med Biol 32, 157-164 (2005).
12. Park, J. W. et al. Development of anti-p185HER2 immunoliposomes for cancer therapy. Proc. Natl. Acad. Sci. USA 92, 1327-1331 (1995).
13. Weiner, L. M. & Carter, P. Tunable antibodies. Nat Biotechnol 23, 556-557 (2005).
14. Woodle, M. C. & Lasic, D. D. Sterically stabilized liposomes. Biochim. Biophys. Acta 1113, 171-199 (1992).
15. Hansen, C. B., Kao, G. Y., Moase, E. H., Zalipsky, S. & Allen, T. M. Attachment of antibodies to sterically stabilized liposomes: evaluation, comparison and optimization of coupling procedures. Biochim. Biophys. Acta 1239, 133-144 (1995).
16. Chiu, G. N., Bally, M. B. & Mayer, L. D. Targeting of antibody conjugated, phosphatidylserine-containing liposomes to vascular cell adhesion molecule 1 for controlled thrombogenesis. Biochim. Biophys. Acta 1613, 115-121 (2003).
17. Hope, M. J., Bally, M. B., Webb, G. & Cullis, P. R. Production of large unilamellar vesicles by a rapid extrusion procedure. Characterization of size distribution, trapped volume and ability to maintain a membrane potential. Biochim. Biophys. Acta 812, 55-65 (1985).
18. Lu, Y., Zi, X., Zhao, Y., Mascarenhas, D. & Pollak, M. Insulin-like growth factor-I receptor signaling and resistance to trastuzumab (Herceptin). J Natl Cancer Inst 93, 1852-1857 (2001).
19. Vivanco, I. & Sawyers, C. L. The phosphatidylinositol 3-Kinase AKT pathway in human cancer. Nat Rev Cancer 2, 489-501 (2002).
20. Knuefermann, C. et al. HER2/PI-3K/Akt activation leads to a multidrug resistance in human breast adenocarcinoma cells. Oncogene 22, 3205-3212 (2003).
21. Clark, A. S., West, K., Streicher, S. & Dennis, P. A. Constitutive and inducible Akt activity promotes resistance to chemotherapy, trastuzumab, or tamoxifen in breast cancer cells. Mol Cancer Ther 1, 707-717 (2002).
22. Ozes, O, N. et al. NF-kappaB activation by tumour necrosis factor requires the Akt serine-threonine kinase. Nature 401, 82-85 (1999).
23. Romashkova, J. A. & Makarov, S. S, NF-kappaB is a target of AKT in anti-apoptotic PDGF signalling. Nature 401, 86-90 (1999).
24. Pommier, Y., Sordet, O., Antony, S., Hayward, R. L. & Kohn, K. W. Apoptosis defects and chemotherapy resistance: molecular interaction maps and networks. Oncogene 23, 2934-2949 (2004).
25. Li, H. et al. The CD20 calcium channel is localized to microvilli and constitutively associated with membrane rafts: antibody binding increases the affinity of the association through an epitope-dependent cross-linking-independent mechanism. J Biol Chem 279, 19893-19901 (2004).
26. Sheikh, M. S., Garcia, M., Pujol, P., Fontana, J. A. & Rochefort, H. Why are estrogen-receptor-negative breast cancers more aggressive than the estrogen-receptor-positive breast cancers? Invasion Metastasis 14, 329-336 (1994).
27. Clynes, R. A., Towers, T. L., Presta, L. G. & Ravetch, J. V. Inhibitory Fc receptors modulate in vivo cytoxicity against tumor targets. Nat Med 6, 443-446 (2000).
28. Warburton, C. et al. Treatment of HER-2/neu overexpressing breast cancer xenograft models with trastuzumab (Herceptin) and gefitinib (ZD1839): drug combination effects on tumor growth, HER-2/neu and epidermal growth factor receptor expression, and viable hypoxic cell fraction. Clin Cancer Res 10, 2512-2524 (2004).
29. Vermette, P., Griesser, H. J., Kambouris, P. & Meagher, L. Characterization of surface-immobilized layers of intact liposomes. Biomacromolecules 5, 1496-1502 (2004).
30. Noppl-Simson, D. A. & Needham, D. Avidin-biotin interactions at vesicles surfaces: adsorption and binding, cross-bridge formation, and lateral interactions. Biophys. J. 70, 1391-1401 (1996).
31. Ghetie, M. A. et al. Homodimerization of tumor-reactive monoclonal antibodies markedly increases their ability to induce growth arrest or apoptosis of tumor cells. Proc. Natl. Acad. Sci. USA 94, 7509-7514 (1997).
32. Pham, L. V., Tamayo, A. T., Yoshimura, L. C., Lo, P. & Ford, R. J. Inhibition of constitutive NF-kappa B activation in mantle cell lymphoma B cells leads to induction of cell cycle arrest and apoptosis. J Immunol 171, 88-95 (2003).
33. Minn, A. J., Rudin, C. M., Boise, L. H. & Thompson, C. B. Expression of bcl-xL can confer a multidrug resistance phenotype. Blood 86, 1903-1910 (1995).
34. Chen, C., Edelstein, L. C. & Gelinas, C. The Rel/NF-kappaB family directly activates expression of the apoptosis inhibitor Bcl-x(L). Mol Cell Biol 20, 2687-2695 (2000).
35. Amundson, S. A. et al. An informatics approach identifying markers of chemosensitivity in human cancer cell lines. Cancer Res 60, 6101-6110 (2000).
36. Vogel, C. L. & Tan-Chiu, E. Trastuzumab plus chemotherapy: convincing survival benefit or not? J Clin Oncol 23, 4247-4250. Epub 2005 May 4223 (2005).
37. Avivi, I., Robinson, S. & Goldstone, A. Clinical use of rituximab in haematological malignancies. Br J Cancer 89, 1389-1394 (2003).
38. Lasic, D. D. & Papahadjopoulos, D. (eds.) Medical applications of liposomes. (Elsevier, New York; 1998).
39. Mayer, L. D., Hope, M. J. & Cullis, P. R Vesicles of variable sizes produced by a rapid extrusion procedure. Biochim. Biophys. Acta 858, 161-168 (1986).

What is claimed is:

1. A method for the identification of therapeutically active antibodies comprising:
(a) formulating a multivalent antibody construct comprising a plurality of antibodies or antibody fragments;
(b) measuring the therapeutic activity of said construct in vitro; and
(c) comparing the in vitro activity of said construct to the in vitro activity of said antibodies or antibody fragments at an equivalent dosage in free form.

2. The method as defined in claim 1, wherein said formulating comprises attaching said antibodies or antibody fragments to the surface of a liposome to form an antibody-liposome conjugate.

3. The method as defined in claim 1, wherein said measuring comprises measuring one or more therapeutic activity parameters in a cell-based screening assay.

4. The method as defined in claims 3, wherein said cell-based screening assay comprises a cell line expressing an antigen specific to said antibodies or said antibody fragments.

5. The method as defined in claim 4, wherein said cell line is a cancer cell line or immune cell line.

6. The method as defined in claim 3, wherein said therapeutic activity parameters are selected from the group consisting of cytotoxicity, cytostasis, apoptosis induction, cell morphology, cytokine production, signal transduction, immune cell activity, cellular proliferation, cellular activation and protein expression and activation.

7. The method as defined in claim 3, wherein said assay is selected from the group consisting of a MTT assay, a XTT assay, and a trypan blue exclusion assay.

8. The method as defined in claim 1, wherein at least one of said antibodies is a monoclonal antibody.

9. The method as defined in claim 1, wherein at least one of said antibody fragments is an antigen-binding single chain antibody, F(ab′)2, Fab, Fd, Fv or scFV fragment.

10. The method as defined in claim 2, wherein the antibody/lipid ratio in said construct is within the range of about 40-75 μg/μmol.

11. The method as defined in claim 10, wherein the number of antibodies per liposome is within the range of about 20 to 50.

12. The method as defined in claim 1, further comprising:
(a) after said comparing, selecting an antibody having increased activity in said construct than in free form in vitro; and
(b) evaluating the effectiveness of said antibody in vivo in an animal model of a disease state.

13. The method as defined in claim 12, wherein said disease state is cancer or an autoimmune disorder.

14. The method as defined in claim 12, wherein said evaluating comprises testing the therapeutic activity of said antibody in free form in vivo.

15. The method as defined in claim 12, wherein said evaluating comprises testing the therapeutic activity of said antibody in conjugated form in vivo.

16. The method as defined in claim 1, wherein measuring said therapeutic activity comprises providing a therapeutic agent separate from said multivalent construct and determining whether said construct and said agent act non-antagonistically.

17. The method as defined in claim 16, wherein said therapeutic agent is a chemotherapeutic agent.

18. The method as defined in claim 12, wherein said evaluating comprises testing the therapeutic activity of said antibodies and said therapeutic agent in combination in vivo.

19. The method as defined in claim 18, further comprising encapsulating said therapeutic agent in said liposome.

20. The method as defined in claim 19, wherein said therapeutic agent is a chemotherapeutic drug.

21. A therapeutic multivalent antibody construct comprising:
(a) a liposome; and
(b) a plurality of antibodies or antibody fragments conjugated to said liposome, wherein said construct enhances the therapeutic activity of said antibodies or fragments in vivo in comparison to said antibodies or fragments at an equivalent dosage in free form.

22. The construct as defined in claim 21, wherein said antibodies are monoclonal antibodies.

23. The construct as defined in claim 22, wherein at least some of said antibodies comprise rituximab.

24. The construct as defined in claim 22, wherein all of said antibodies comprise rituximab.

25. The construct as defined in claim 21, wherein said construct enhances crosslinking of target antigens.

26. The construct as defined in claim 21, wherein at least some of said antibodies or antibody fragments target different antigens.

27. The construct as defined in claim 21, wherein said antibody fragment is an antigen-binding, single chain antibody, F(ab′)2, Fab, Fd, Fv or scFV fragment.

28. The construct as defined in claim 21, further comprising a therapeutic agent encapsulated within said liposome.

29. The construct as defined in claim 28, wherein said therapeutic agent is a chemotherapeutic anticancer drug.

30. The construct as defined in claim 29 wherein said drug and said antibodies produce a non-antagonistic therapeutic effect in vivo when administered in combination.

31. The construct as defined in claim 21, wherein the antibody/lipid ratio in said construct is within the range of about 40-75 μg/μmol.

32. The method as defined in claim 31, wherein the number of antibodies per liposome is within the range of about 20 to 50.

33. The construct as defined in claim 21, wherein said liposome comprises lipids selected from the group consisting of phosphoglycerides and sphingolipids.

34. The construct as defined in claim 33, wherein said lipids are selected from the group consisting of phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine and dilinoleoylphosphatidylcholine.

35. The use of a construct as defined in claim 21 for cancer or autoimmune disorder therapy.

36. A composition comprising a plurality of antibodies or antibody fragments conjugated to the surface of a liposome, wherein the anticancer therapeutic activity of said composition in vivo is enhanced in comparison to an equivalent dosage of said antibodies or said fragments in free form.

37. The composition as defined in claim 36, further comprising a therapeutic agent separate from said antibodies or antibody fragments encapsulated within said liposome.

38. The composition as defined in claim 37, wherein said therapeutic agent acts synergistically with said antibodies or said antibody fragments.

39. The composition as defined in claim 36, wherein said composition enhances crosslinking of target antigens.

40. The composition as defined in claim 36, wherein at least some of said antibodies target different antigens.
